(19) United States
(12) Patent Application Publication
(10) Pub. No.: US 2015/0374344 A1
(43) Pub. Date: Dec. 31, 2015

(54) ULTRASONIC DIAGNOSTIC APPARATUS AND PROGRAM

(71) Applicant: GE Medical Systems Global Technology Company LLC, Waukesha, WI (US)

(72) Inventors: Tetsuo KOIDE, Tokyo (JP); Hiroshi HASHIMOTO, Tokyo (JP); Masashi SEKI, Tokyo (JP)

(21) Appl. No.: 14/788,234

(22) Filed: Jun. 30, 2015

(30) Foreign Application Priority Data
Jun. 30, 2014 (JP) 2014-133452

(51) Int. Cl. A61B 8/08; A61B 8/00; A61B 8/14

(52) U.S. Cl. CPC A61B 8/5246; A61B 8/14; A61B 8/4254; A61B 8/463; A61B 8/466; A61B 8/54

(57) ABSTRACT

An ultrasonic diagnostic apparatus comprising an image display control section for displaying a first image representing positions of first ultrasonic scan planes at certain intervals of time based on information on positions of the first scan planes at certain intervals of time stored in a storage section, and displaying, based on detected information from a movement detecting section, a second image representing positions of second scan planes at certain intervals of time formed by performing a second 3D ultrasonic scan on a subject by an operator moving the ultrasonic probe, the image display control section displaying the first and second images so that the first scan planes and second scan planes have mutually corresponding positional relationships.

### FIELD OF THE INVENTION

Embodiments of the present invention relate to an ultrasonic diagnostic apparatus and a program for performing an ultrasonic scan in a three-dimensional (3D) manner by an operator moving an ultrasonic probe.

### BACKGROUND

In ultrasonic diagnostic apparatuses, ultrasound is transmitted from an ultrasonic probe to the inside of a subject, and echo signals reflected from the inside of the subject are received by the ultrasonic probe. Then, an ultrasonic image such as a B-mode image is produced based on the received echo signals and displayed.

In some of such ultrasonic diagnostic apparatuses, transmission/reception of ultrasound is performed on a 3D region to acquire ultrasonic volume data. The volume data is in some cases acquired by a 3D probe, such as a mechanical 3D probe, and in other cases, an operator changes an angle of the ultrasonic probe or moves the ultrasonic probe to perform transmission/reception of ultrasound on a 3D region and acquire ultrasonic volume data (see Patent Document 1, Japanese Patent No. 4137516, for example).

### SUMMARY OF THE INVENTION

When an operator moves an ultrasonic probe to acquire ultrasonic volume data as described above, an inexperienced operator, among others, is inexpert in an operation of moving the ultrasonic probe, and may move the ultrasonic probe too fast or too slow, for example, as compared with a skilled operator. Moreover, an inexperienced operator may perform an ultrasonic scan on a region different from that on which a skilled operator would otherwise perform a scan. Thus, there is a need for means for supporting an inexperienced operator in case that an operator performs a 3D ultrasonic scan by moving an ultrasonic probe.

The invention in one aspect made for solving the problem described above is an ultrasonic diagnostic apparatus characterized in comprising: an ultrasonic probe for performing transmission/reception of ultrasound on a subject; a movement detecting section for detecting movement of an ultrasonic scan plane by said ultrasonic probe; a storage section for storing therein information on positions of a plurality of first scan planes in a first three-dimensional (3D) ultrasonic scan on said subject; and an image display control section for displaying in a display section a first image representing positions of said first scan planes based on said information on positions stored in said storage section, and displaying in said display section a second image representing positions of a plurality of second scan planes formed by performing a second 3D ultrasonic scan on said subject by an operator moving said ultrasonic probe, based on detected information from said movement detecting section, said image display control section displaying said first and second images so that said first scan planes and second scan planes have mutually corresponding positional relationships.

According to the invention in the aspect described above, the first and second images are displayed so that the first scan planes and second scan planes have mutually corresponding positional relationships. For example, when information on positions of the first scan planes at certain intervals of time corresponds to ideal movement of the ultrasonic probe, and information on positions of the second scan planes at certain intervals of time is associated with movement of the ultrasonic probe by an inexperienced operator, the inexperienced operator can learn ideal movement of the ultrasonic probe by comparing the first image with the second image.

### BRIEF DESCRIPTION OF THE DRAWINGS

FIG. 1 is a block diagram showing an exemplary schematic configuration of an ultrasonic diagnostic apparatus in a first embodiment of the present invention.

FIG. 2 is a block diagram showing a configuration of a display processing section in the ultrasonic diagnostic apparatus shown in FIG. 1.

FIG. 3 is a diagram showing a first image displayed in a display section.

FIG. 4 is a diagram for explaining a time separation between first scan planes adjacent to each other.

FIG. 5 is a diagram showing the display section in which a second image is displayed along with the first image.

FIG. 6 is a diagram for explaining a time separation between the first scan planes adjacent to each other and that between second scan planes adjacent to each other.

FIG. 7 is a diagram showing the display section in which the first image including a first 3D region image and the second image including a second 3D region image are displayed.

FIG. 8 is a diagram showing the display section in which a warning image is displayed.

FIG. 9 is a diagram showing the display section in which a guide image is displayed.

FIG. 10 is a block diagram showing a configuration of the display processing section in a second variation of the first embodiment.

FIG. 11 is a diagram for explaining an example of a distance between the first and second scan planes in the second variation of the first embodiment.

FIG. 12 is a diagram showing an example of an overlapping portion of the first and second scan planes when one of them is projected onto the other in the second variation of the first embodiment.

FIG. 13 is a diagram showing the display section in which a warning image is displayed in the second variation of the first embodiment.

FIG. 14 is a block diagram showing a function of a control section in a third variation of the first embodiment.

FIG. 15 is a block diagram showing an exemplary schematic configuration of an ultrasonic diagnostic apparatus in a second embodiment of the present invention.

FIG. 16 is a block diagram showing a configuration of a display processing section in the ultrasonic diagnostic apparatus shown in FIG. 15.

FIG. 17 is a block diagram showing an exemplary schematic configuration of an ultrasonic diagnostic apparatus in a third embodiment of the present invention.

FIG. 18 is a diagram showing the display section in which velocity variation images are displayed along with the first and second images.

FIG. 19 is an enlarged view of the velocity variation images shown in FIG. 18.

FIG. 20 is a diagram for explaining movement of an ultrasonic probe.

FIG. 21 is a diagram showing the velocity variation images including numeric values.

### DETAILED DESCRIPTION

Now embodiments of the present invention will be described with reference to the accompanying drawings.

### First Embodiment

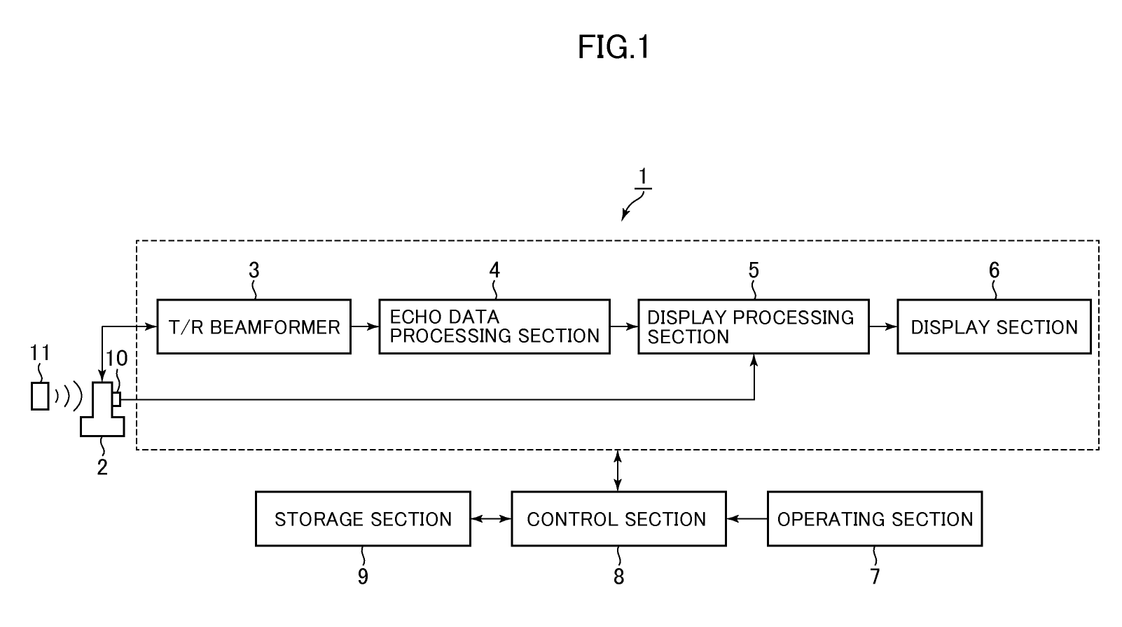

To begin with, a first embodiment will be described. An ultrasonic diagnostic apparatus 1 shown in FIG. 1 comprises an ultrasonic probe 2, a transmission/reception (T/R) beamformer 3, an echo data processing section 4, a display processing section 5, a display section 6, an operating section 7, a control section 8, and a storage section 9.

The ultrasonic probe 2 is configured to comprise a plurality of ultrasonic vibrators (not shown) arranged in an array, and ultrasound is transmitted to a subject and echo signals thereof are received by the ultrasonic vibrators.

The ultrasonic probe 2 is provided with a magnetic sensor 10 comprised of a Hall element, for example. By the magnetic sensor 10, magnetism generated from a magnetism generating section 11 comprised of a magnetism-generating coil, for example, is detected. A detection signal from the magnetic sensor 10 is input to the display processing section 5. The detection signal from the magnetic sensor 10 may be input to the display processing section 5 via a cable, which is not shown, or wirelessly input to the display processing section 5. The magnetism generating section 11 and magnetic sensor 10 are provided for detecting a position of the ultrasonic probe 2 (including a tilt of the ultrasonic probe 2), as described later. The magnetic sensor 10 represents an exemplary embodiment of the magnetism detecting section in the present invention. The magnetism generating section 11 represents an exemplary embodiment of the magnetism generating section in the present invention.

The T/R beamformer 3 supplies an electric signal to the ultrasonic probe 2 for transmitting ultrasound from the ultrasonic probe 2 with specified scan conditions based on a control signal from the control section 8. The T/R beamformer 3 also applies signal processing such as A/D conversion and phased addition processing to echo signals received by the ultrasonic probe 2, and outputs echo data after the signal processing to the echo data processing section 4.

The echo data processing section 4 applies processing for producing an ultrasonic image to the echo data output from the T/R beamformer 3. For example, the echo data processing section 4 applies B-mode processing such as logarithmic compression processing and envelope detection processing, and creates B-mode data.

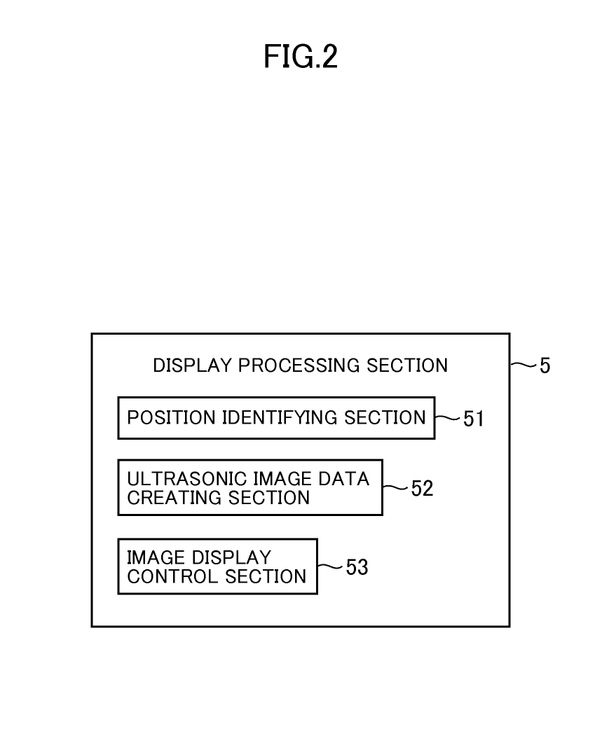

The display processing section 5 comprises a position identifying section 51, an ultrasonic image data creating section 52, and an image display control section 53, as shown in FIG. 2. The position identifying section 51 calculates information on a position of the ultrasonic probe 2 (which will be referred to hereinbelow as “information on a probe position”) in a 3D space coordinate system whose origin lies at the magnetism generating section 11 based on a magnetism detection signal from the magnetic sensor 10. Further, the position identifying section 51 calculates information on a position of an echo signal in the 3D space coordinate system based on the information on a probe position. The calculation of the information on a position enables information on positions of ultrasonic scan planes by the ultrasonic probe 2 to be identified in the 3D space coordinate system. The magnetic sensor 10 represents an exemplary embodiment of the magnetism detecting section in the present invention. The magnetism generating section 11 represents an exemplary embodiment of the magnetism generating section in the present invention. The position identifying section 51 represents an exemplary embodiment of the position identifying section in the present invention. The 3D space coordinate system whose origin lies at the magnetism generating section 11 represents an exemplary embodiment of the coordinate system whose origin lies at a specified point in the present invention.

The ultrasonic image data creating section 52 scan-converts the data supplied from the echo data processing section 4 by a scan converter to create ultrasonic image data. For example, the ultrasonic image data creating section 52 scan-converts B-mode data to create B-mode image data. The data before the scan conversion by the scan converter is sometimes referred to as raw data.

The image display control section 53 displays an ultrasonic image in the display section 6 based on the ultrasonic image data. The ultrasonic image is a B-mode image, for example, based on the B-mode image data.

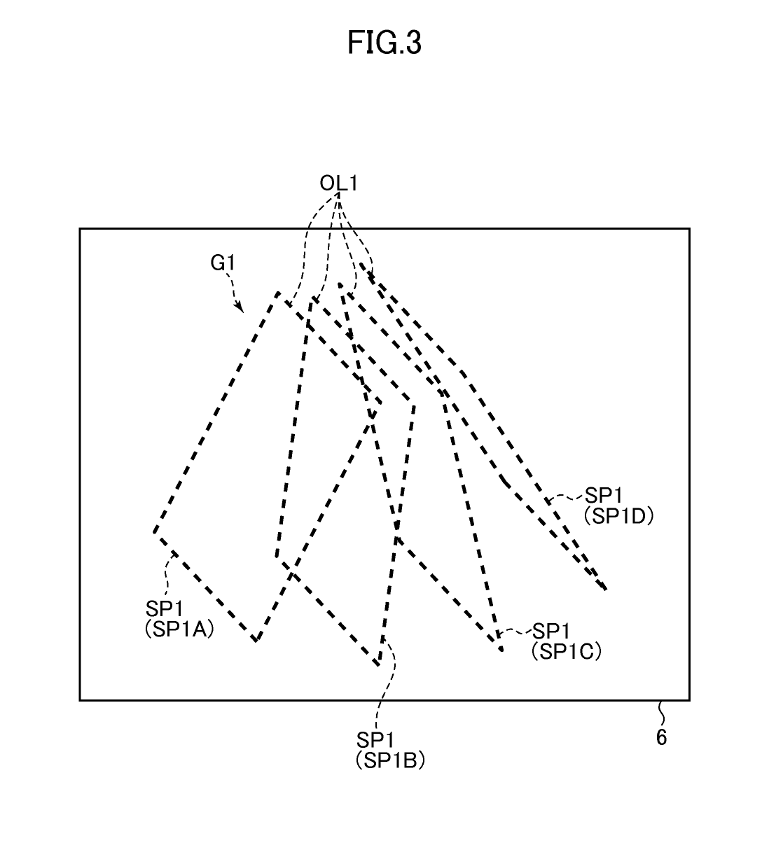

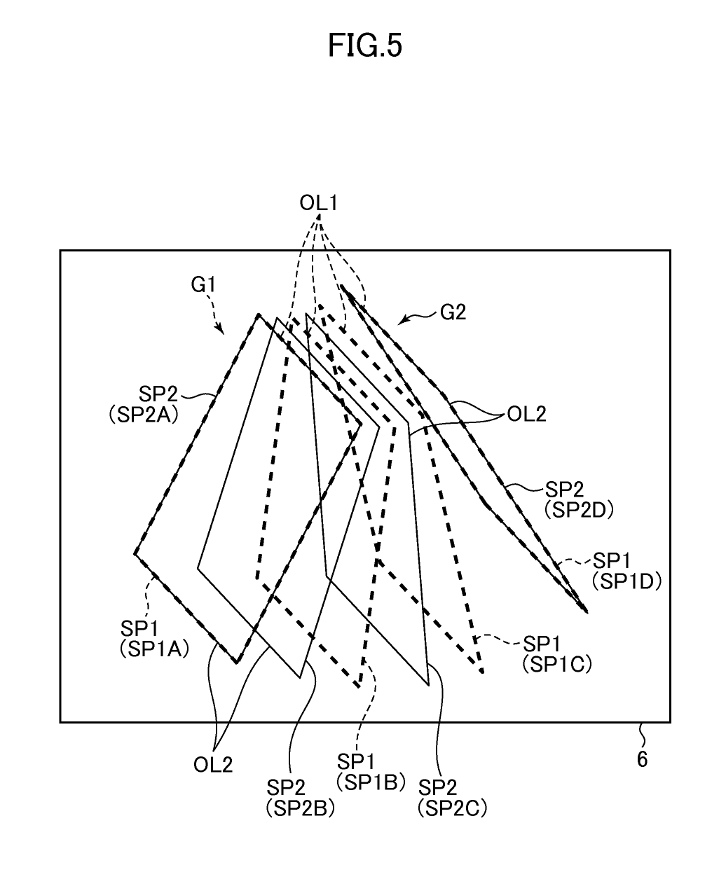

The image display control section 53 also displays a first image G1 representing positions of first scan planes SP1 at certain intervals of time in the display section 6, as shown in FIGS. 3, 5, etc. which will be described later. The image display control section 53 moreover displays a second image G2 in the display section 6 representing positions of second scan ultrasonic planes SP2 at certain intervals of time, the second scan plane having its position changing associated with movement of the ultrasonic probe 2 in a 3D region. Details thereof will be discussed later. The 3D region is a region in a 3D space coordinate system whose origin lies at the magnetism generating section 11. The image display control section 53 represents an exemplary embodiment of the image display control section in the present invention.

The display section 6 is an LCD (Liquid Crystal Display), a CRT (Cathode Ray Tube), or the like. The operating section 7 is configured to comprise a keyboard for allowing an operator to input a command and/or information, a pointing device, and the like (not shown).

The control section 8 is a processor such as a CPU (Central Processing Unit). The control section 8 loads thereon a program stored in the storage section 9 and controls several sections in the ultrasonic diagnostic apparatus 1. For example, the control section 8 loads thereon a program stored in the storage section 9 and executes functions of the T/R beamformer 3, echo data processing section 4, and display processing section 5 by the loaded program.

The control section 8 may execute all of the functions of the T/R beamformer 3, all of the functions of the echo data processing section 4, and all of the functions of the display processing section 5 by the program, or execute only some of the functions by the program. In case that the control section 8 executes only some of the functions, the remaining functions may be executed by hardware such as circuitry.

It should be noted that the functions of the T/R beamformer 3, echo data processing section 4, and display processing section 5 may be implemented by hardware such as circuitry.

The storage section 9 is an HDD (Hard Disk Drive), and/or a semiconductor memory such as a RAM (Random Access Memory) and/or a ROM (Read-Only Memory). In addition to the control program, the storage section 9 stores therein the information on positions of the first scan planes SP1 at certain intervals of time. The information on positions of the first scan planes at certain intervals of time may be information on positions in the 3D space coordinate system whose origin lies at the magnetism generating section 11, or information on relative positions identifying a positional relationship among a plurality of the first scan planes with one another.

Now an operation of the ultrasonic diagnostic apparatus 1 in the present embodiment will be described below. First, a skilled operator performs an ultrasonic scan on a specified region in a subject P by the ultrasonic probe 2. The skilled operator will be referred to hereinbelow as first operator OP1. The first operator OP1 moves the ultrasonic probe 2 to perform an ultrasonic scan for a plurality of scan planes in a 3D region. The scan planes by the first operator OP1 will be referred to hereinbelow as first scan planes SP1. The ultrasonic scan by the first operator OP1 will be referred to hereinbelow as first 3D ultrasonic scan.

Data based on echo signals acquired by the ultrasonic probe 2 is stored in the storage section 9. The data stored in the storage section 9 may be raw data or ultrasonic image data. In addition to the data based on echo signals, the storage section 9 stores therein information on a position of the data based on echo signals. The information on a position is that obtained by the position identifying section 51, which is information on a position in a 3D space coordinate system whose origin lies at the magnetism generating section 11. The information on a position is stored along with information on a time regarding an acquisition time for the echo signals. Thus, information on positions of a plurality of first scan planes SP1 in the 3D region at certain intervals of time is stored.

Next, an inexperienced operator performs transmission/reception of ultrasound on the same region in the subject P as that on which the first operator OP1 has performed transmission/reception of ultrasound. The inexperienced operator will be referred to hereinbelow as second operator OP2.

Specifically, the second operator OP2 first performs transmission/reception of ultrasound by the ultrasonic probe 2 for the same plane in the subject P as a scan plane SP1 for which the first operator OP1 started transmission/reception of ultrasound. The second operator OP2 decides whether the plane is identical or not while observing a real-time B-mode image based on echo signals obtained at the ultrasonic probe 2, for example.

After the plane is decided to be identical, the second operator OP2 starts transmission/reception of ultrasound for a plurality of scan planes in a 3D region while moving the ultrasonic probe 2. Before starting transmission/reception of ultrasound for the 3D region, the second operator OP2 presses a start button at the operating section 7. Upon pressing of the start button, the image display control section 53 displays a first image G1 representing positions of the first scan planes SP1 at certain intervals of time t in the display section 6, as shown in FIG. 3. The first image G1 includes first outlines OL1 representing outlines of the first scan planes SP1.

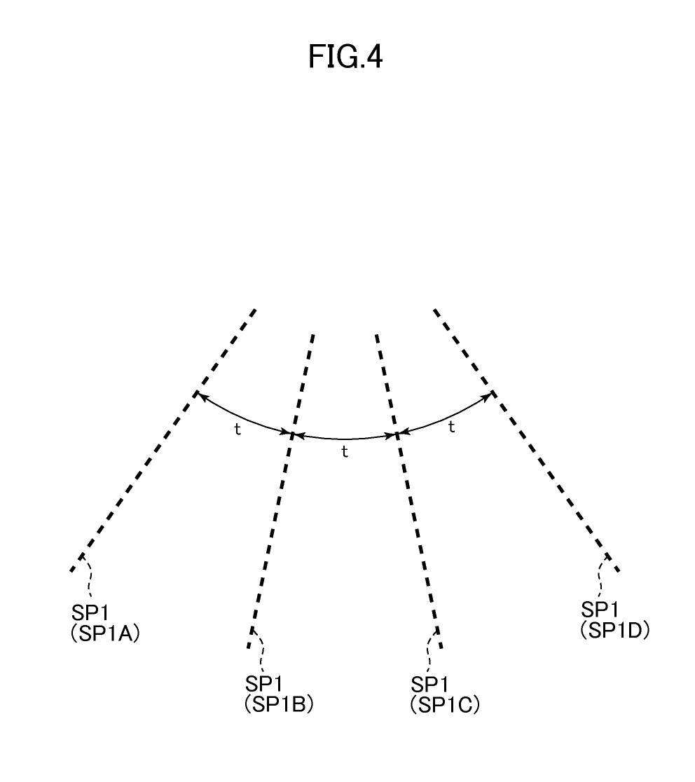

In FIG. 3, four first scan planes SP1A-SP1D are shown as the first scan planes SP1. A first scan plane SP1A is a plane for which the first operator OP1 started an ultrasonic scan. The first scan plane SP1A represents an exemplary embodiment of the first scan-start plane in the present invention. As shown in FIG. 4, a time separation between the first scan planes SP1 adjacent to each other is the certain period of time t.

The image display control section 53 displays the first scan planes SP1A-SP1D so that they have a positional relationship identified by the information on positions of the first scan planes SP1 at certain intervals of time t stored in the storage section 9.

Once the first image G1 has been displayed, the second operator OP2 performs transmission/reception of ultrasound for a plurality of scan planes in the 3D region while moving the ultrasonic probe 2. The scan planes by the second operator OP2 will be referred to hereinbelow as second scan planes SP2. The ultrasonic scan by the second operator OP2 will be referred to hereinbelow as second 3D ultrasonic scan.

The image display control section 53 displays in the display section 6 a second image G2 representing positions of the second scan planes SP2 at certain intervals of time t, the second scan plane having its position changing associated with movement of the ultrasonic probe 2, as shown in FIG. 5. The image display control section 53 displays the second image G2 based on information on positions of the scan planes identified by the position identifying section 51. The second image G2 includes second outlines OL2 representing outlines of the second scan planes SP2. The second outlines OL2 may be displayed in a color different from that of the first outlines OL1. While the first outlines OL1 are represented in dashed lines in the drawings, they may be represented in solid lines.

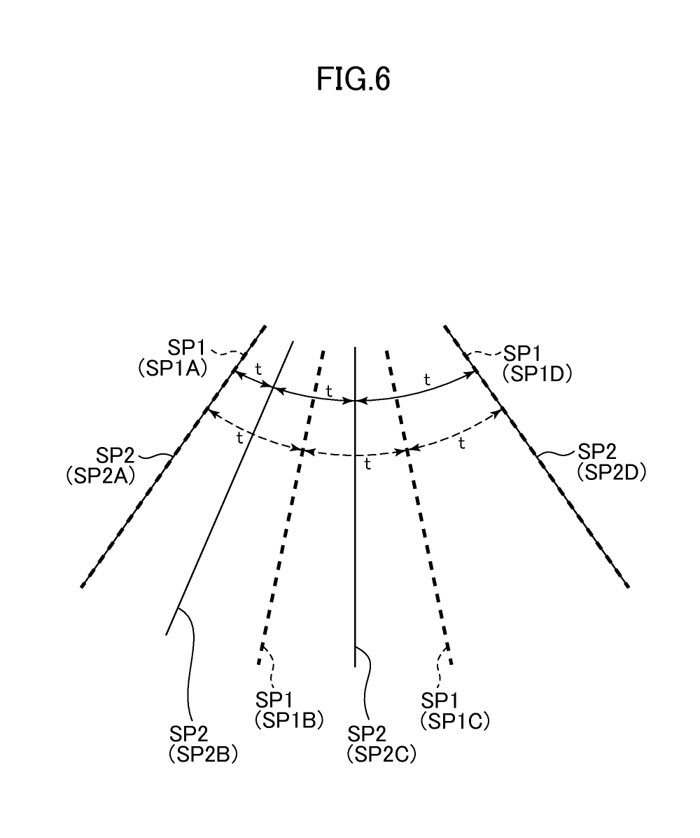

In FIG. 5, four second scan planes SP2A-SP2D are shown as the second scan planes SP2. A second scan plane SP2A is a plane for which the second operator OP2 started an ultrasonic scan. The second scan plane SP2A represents an exemplary embodiment of the second scan-start plane in the present invention. The image display control section 53 displays the first image G1 and second image G2 so that the first scan planes SP1 and second scan planes SP2 at certain intervals of time t have mutually corresponding positional relationships. In particular, a pair of the first scan plane SP1A and second scan plane SP2A, a pair of the first scan plane SP1B and second scan plane SP2B, a pair of the first scan plane SP1C and second scan plane SP2C, and a pair of the first scan plane SP1D and second scan plane SP2D each represent scan planes at an identical time. Referring to FIG. 6, a time separation between first scan planes SP1 adjacent to each other is a certain period of time t, and a time separation between second scan planes SP2 adjacent to each other is the certain period of time t.

It should be noted that the position of the subject P may be changed or unchanged from a time of the transmission/reception of ultrasound by the first operator OP1 to a time of the transmission/reception of ultrasound by the second operator OP2. In case that the position of the subject P is unchanged, an identical portion in the subject P is located at the same coordinates in the coordinate system whose origin lies at the magnetism generating section 11 both in the information on positions of the first scan planes SP1 stored in the storage section 9 and real-time information on positions of the second scan planes SP2 identified by the position identifying section 51. Therefore, the image display control section 53 displays the first image G1 and second image G2 based on the positions of the first scan planes SP1 and second scan planes SP2 identified in the coordinate system whose origin lies at the magnetism generating section 11 according to the information on positions stored in the storage section 9 and that obtained by the position identifying section 51. Thus, the first scan planes SP1 and second scan planes SP2 can be displayed so that they have mutually corresponding positional relationships.

On the other hand, even when the position of the subject P has changed, the image display control section 53 can display the first scan planes SP1 and second scan planes SP2 so that they have mutually corresponding positional relationships. In particular, based on the information on positions stored in the storage section 9, a relative positional relationship among a plurality of the first scan planes SP1 can be identified. Moreover, based on the information on positions of the second scan planes SP2 identified by the position identifying section 51, a relative positional relationship among a plurality of the second scan plane SP2 can be identified.

Therefore, for example, when the position of the first scan plane SP1A, which is the scan-start plane among a plurality of the first scan planes SP1, and the position of the second scan plane SP2A, which is the scan-start plane among a plurality of the second scan planes SP2, are identical in the subject, the image display control section 53 displays the first scan planes SP1 and second scan planes SP2 based on the information on positions stored in the storage section 9 and information on positions identified by the position identifying section 51, whereby it can display the first scan planes SP1 and second scan planes SP2 so that they have mutually corresponding positional relationships.

More particularly, the image display control section 53 displays the first scan plane SP1A and second scan plane SP2A at the same positions in the display section 6. Moreover, the image display control section 53 displays the first scan planes SP1B, SP1C, SP other than the first scan plane SP1A in the display section 6 based on a relative positional relationship among the first scan planes SP1A-SP1D identified from the information on positions stored in the storage section 9. The image display control section 53 also displays the second scan planes SP2B, SP2C, SP2D other than the second scan plane SP2A in the display section 6 based on a relative positional relationship among the second scan planes SP2A-SP2D identified from the information on positions obtained by the position identifying section 51. Thus, the first scan planes SP1 and second scan planes SP2 can be displayed so that they have mutually corresponding positional relationships.

In FIGS. 5 and 6, among the first scan planes SP1 and second scan planes SP2 at respective identical times, a pair of the first scan plane SP1A and second scan plane SP2A, and a pair of the first scan plane SP1D and the second scan plane SP2D each lie at an identical position; however, the other pairs of the first scan planes SP1 and second scan planes SP2 lie different positions. This means that movement of the ultrasonic probe 2 by the first operator OP1 is different from that by the second operator OP2. In particular, the first operator OP1 moves the ultrasonic probe 2 at a constant speed because separations for the first scan planes SP1A-SP1D are equal.

On the other hand, separations for the second scan planes SP2A-SP2D are not equal. The separation between the second scan plane SP2A and second scan plane SP2B is smallest as compared with the others, and the separation becomes larger in sequence: the separation between the second scan plane SP2B and second scan plane SP2C, and next, the separation between the second scan plane SP2C and second scan plane SP2D. This implies that the second operator OP2 cannot move the ultrasonic probe 2 at a constant speed yet, and the speed of movement gradually increases.

Thus, according to the present embodiment, by displaying the first image G1 and second image G2, an inexperienced operator can compare movement of the ultrasonic probe 2 by him/herself with that by a skilled operator to learn ideal movement of the ultrasonic probe 2. Therefore, the inexperienced operator can achieve the same or generally the same movement of the ultrasonic probe 2 as that by the skilled operator by repeatedly performing transmission/reception of ultrasound until the second scan planes SP2 match or generally match the first scan planes SP1 at corresponding times in the first image G1 and second image G2.

It should be noted that in case that an ultrasonic scan is performed for a different region after an ultrasonic scan is performed for a specified region in the subject P, an ultrasonic scan by the second operator OP2 is performed after an ultrasonic scan by the first operator OP1 has been performed again in a similar manner to that described above.

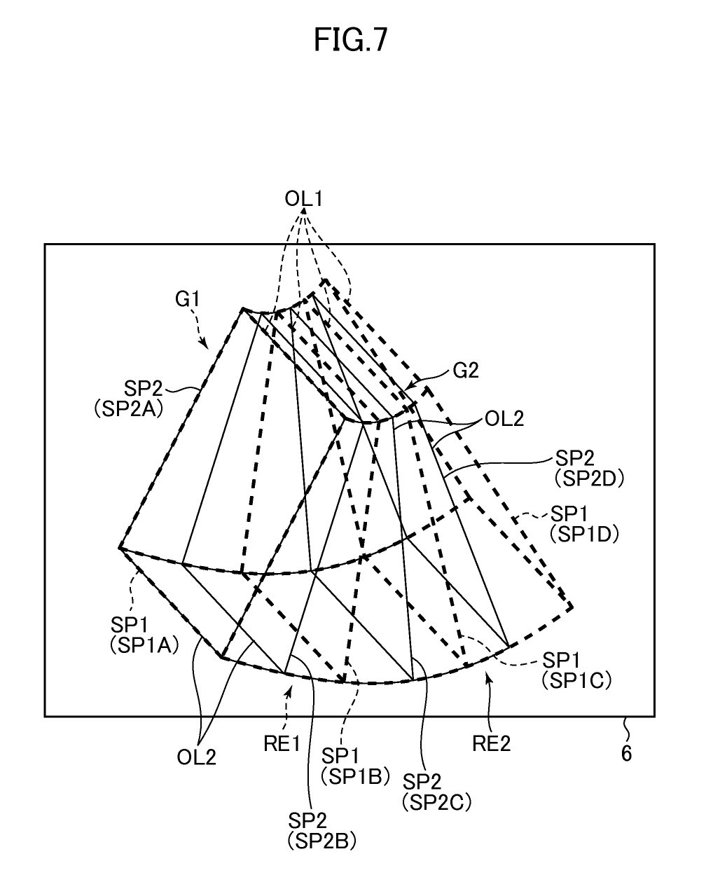

Next, a variation of the first embodiment will be described below. A first variation will be described first. The image display control section 53 may display a first 3D region image RE1 as part of the first image G1, as shown in FIG. 7. The first 3D region image RE1 is a line representing an outline of a region for the first 3D ultrasonic scan by the first operator OP1 (indicated by a dashed line in FIG. 7). The image display control section 53 displays the first 3D region image RE1 based on the information on positions stored in the storage section 9 (information on positions of data based on echo signals obtained by the first operator OP1).

The image display control section 53 may also display a second 3D region image RE2 as part of the second image G2. The second 3D region image RE2 is a line representing an outline of a region for the second 3D ultrasonic scan by the second operator OP2 (indicated by a solid line in FIG. 7). The image display control section 53 displays the image of the second 3D region RE2 based on the information on positions obtained by the position identifying section 51.

In FIG. 7, the region for the ultrasonic scan by the second operator OP2 ranges from the second scan plane SP2A to the second scan plane SP2D. The region scanned by the second operator OP2 ranging from the second scan plane SP2A to the second scan plane SP2D will be referred to hereinbelow as second 3D region.

On the other hand, the region for the ultrasonic scan by the first operator OP1 is a region ranging from the first scan plane SP1A to the first scan plane SP1D. The region scanned by the first operator OP1 ranging from the first scan plane SP1A to the first scan plane SP1D will be referred to hereinbelow as first 3D region.

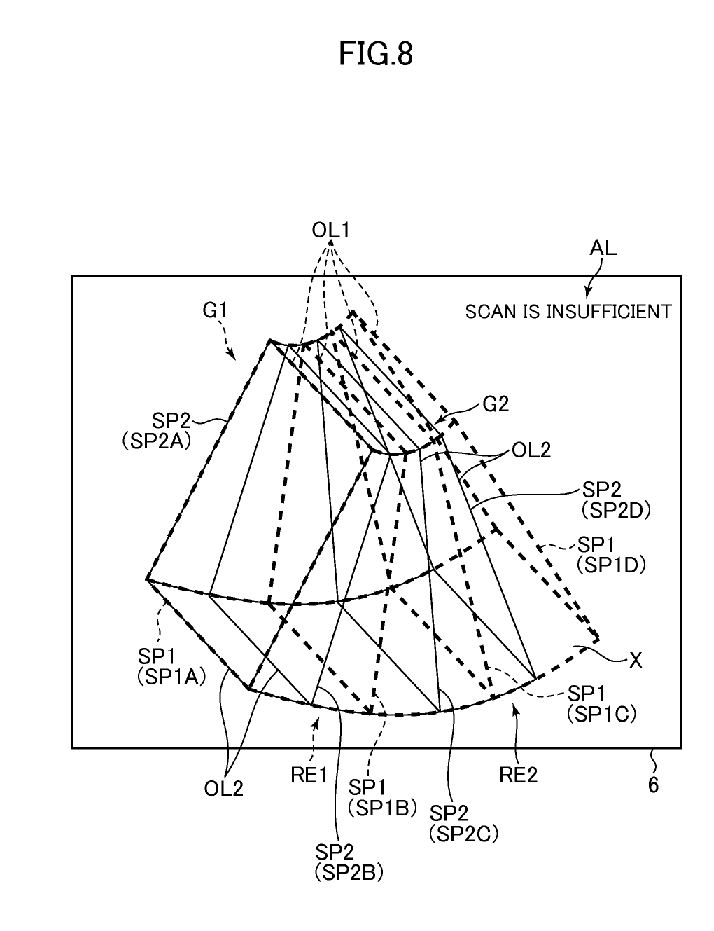

In FIG. 7, the second 3D region is smaller than the first 3D region, and an unscanned region X where no ultrasonic scan is performed by the second operator OP2 exists in the first 3D region. In this case, as shown in FIG. 8, the image display control section 53 may display a warning image AL in the display section 6. The warning image AL is comprised of characters indicating the region for the ultrasonic scan by the second operator OP2 is insufficient relative to the region for the ultrasonic scan by the first operator OP1 and the unscanned region X is left. However, the warning image AL is merely an example and it is not limited to the image comprised of characters shown in FIG. 8. The display section 6 in which the warning image AL is displayed represents an exemplary embodiment of the warning notifying section in the present invention.

It should be noted that sound, in place of the warning image AL, may be output from a speaker in the ultrasonic diagnostic apparatus 1.

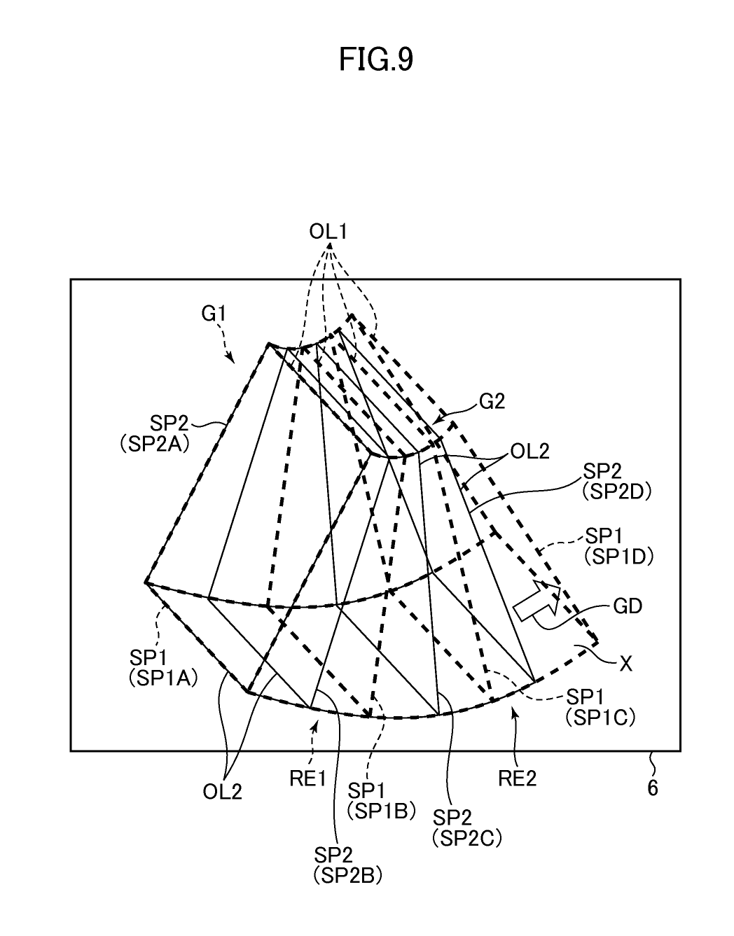

Moreover, when an unscanned region X exists as described above, the image display control section 53 may display a guide image GD in the display section 6, as shown in FIG. 9. The guide image GD is an image of an arrow for guiding the second operator OP2 to an ultrasonic scan by the ultrasonic probe 2 on the unscanned region X. The arrow of the guide image GD indicates a direction in which the ultrasonic probe 2 is to be moved.

The guide image GD may be displayed along with the warning image AL.

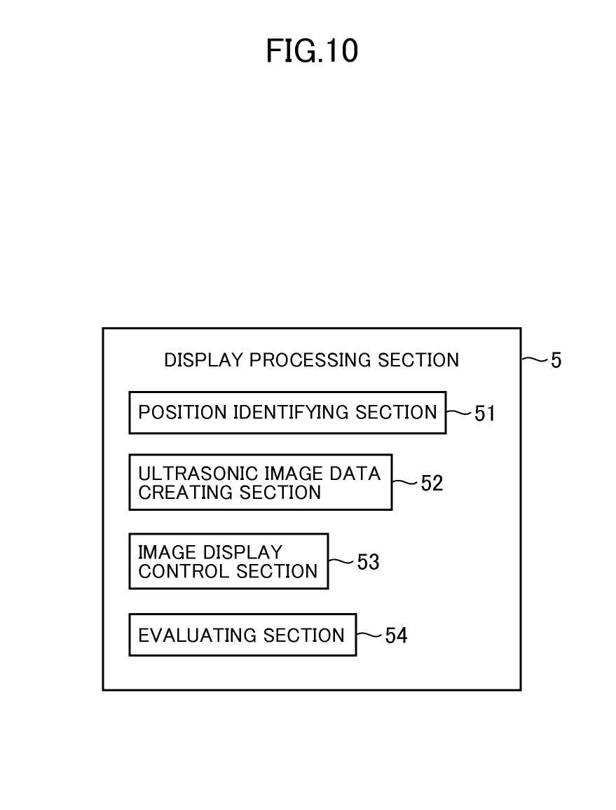

Next, a second variation will be described below. The display processing section 5 comprises an evaluating section 54, in addition to the position identifying section 51, ultrasonic image data creating section 52, and image display control section 53, as shown in FIG. 10. The evaluating section 54 evaluates movement of the ultrasonic probe 2 based on a positional relationship between a first scan plane SP1 and a second scan plane SP2 corresponding in time to each other. The evaluating section 54 represents an exemplary embodiment of the evaluating section in the present invention.

The evaluating section 54 will now be described in more detail. The positional relationship between a first scan plane SP1 and a second scan plane SP2 corresponding in time to each other described above is a distance between the first scan plane SP1 and second scan plane SP2 corresponding in time to each other. The positional relationship may also be a degree of overlapping between the first scan plane SP1 and second scan plane SP2 corresponding in time to each other when one of them is projected onto the other.

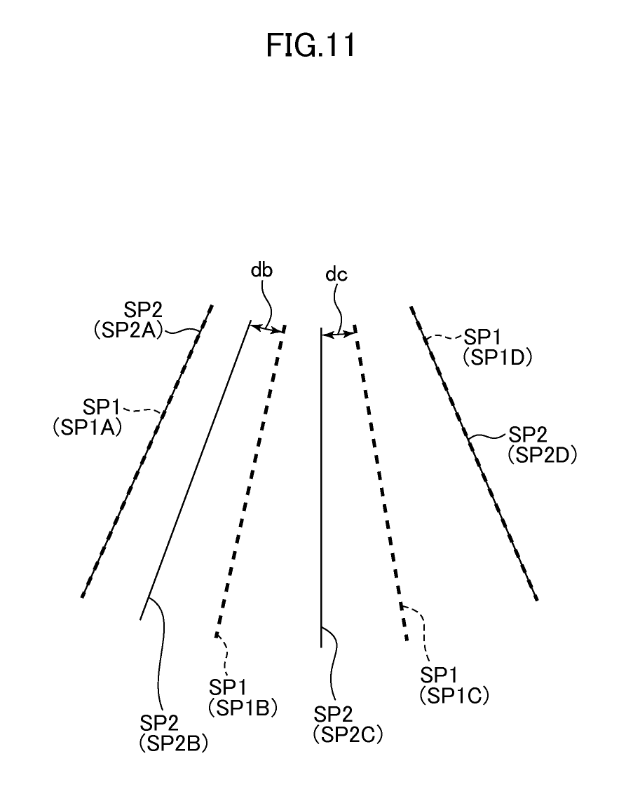

The evaluating section 54 calculates a distance D between the first scan plane SP1 and second scan plane SP2 corresponding in time to each other. The distance D may be a shortest distance between the first scan plane SP1 and second scan plane SP2 corresponding in time to each other. The distance D is calculated based on the information on positions stored in the storage section 9 and information on positions obtained by the position identifying section 51. In particular, the evaluating section 54 calculates a distance between the first scan plane SP1A and second scan plane SP2A, a distance between the first scan plane SP1B and second scan plane SP2B, a distance between the first scan plane SP1C and second scan plane SP2C, and a distance between the first scan plane SP1D and second scan plane SP2D. Referring to FIG. 11, the distance between the first scan plane SP1A and second scan plane SP2A, and the distance between the first scan plane SP1D and second scan plane SP2D are zero. The distance between the first scan plane SP1B and second scan plane SP2B is db, and the distance between the first scan plane SP1C and second scan plane SP2C is dc.

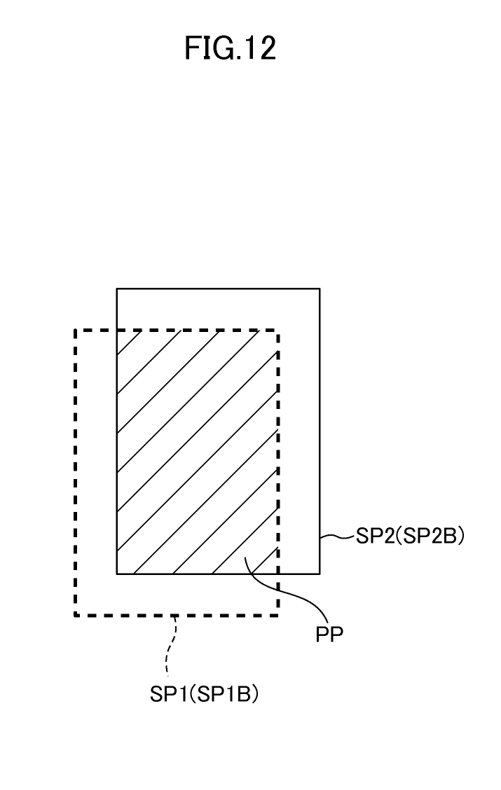

Alternatively, the evaluating section 54 calculates an area S of an overlapping portion of the first scan plane SP1 and second scan plane SP2 corresponding in time to each other. In particular, the evaluating section 54 calculates an area S of an overlapping portion of the first scan plane SP1A and second scan plane SP2A, an area S of an overlapping portion of the first scan plane SP1B and second scan plane SP2B, an area S of an overlapping portion of the first scan plane SP and second scan plane SP2C, and an area S of an overlapping portion of the first scan plane SP1D and second scan plane SP2D. Referring to FIG. 12, an overlapping portion PP (hatched portion) of the first scan plane SP1B and second scan plane SP2B is shown. The evaluating section 54 calculates an area S of the overlapping portion PP.

It should be noted that in case that the first scan plane SP1 and second scan plane SP2 corresponding in time to each other are not coplanar, the evaluating section 54 calculates an area S′ of an overlapping portion of the first scan plane SP1 and second scan plane SP2 by projecting one of them onto the other.

The evaluating section 54 identifies a number N of scan planes for which the distance D is greater than a specified threshold Dth or the area S is smaller than a specified threshold Sth. The threshold Dth is set to a value such that a distance between the first scan plane SP1 and second scan plane SP2 corresponding in time to each other is not too large and it is certain that the ultrasonic scan by the second operator OP2 is performed by generally ideal movement of the ultrasonic probe 2. The threshold Sth is set to a value such that a shift between the first scan plane SP1 and second scan plane SP2 corresponding in time to each other in an in-plane direction is not too large and it is certain that the position of the ultrasonic scan by the second operator OP2 is generally the same position as that of the ultrasonic scan by the first operator OP1.

The evaluating section 54 decides whether the identified number N of the scan planes is equal to or greater than a specified threshold Nth or not. The threshold Nth is set to a value such that the velocity of movement of a scan plane or a scanned position in the second 3D ultrasonic scan by the second operator OP2 is different from that in the first 3D ultrasonic scan by the first operator OP1 and it is certain that the scan should be performed again.

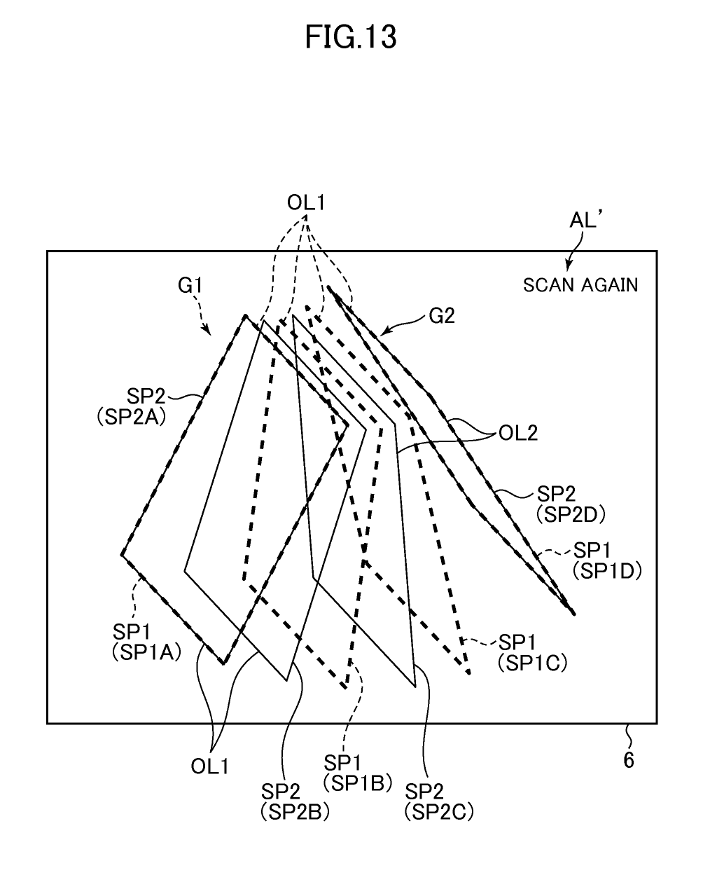

When the number N of the scan planes is decided to be equal to or greater than The threshold Nth by the evaluating section 54, the image display control section 53 displays a warning image AL′ in the display section 6, as shown in FIG. 13. The warning image AL′ is comprised of characters directing the second operator OP2 to perform the ultrasonic scan again because movement of the ultrasonic probe 2 by the second operator OP2 is not ideal. However, the warning image AL′ is merely an example and any means for informing the second operator OP2 of the fact that movement of the ultrasonic probe 2 by him/her is not ideal will work. The display section 6 in which the warning image AL′ is displayed represents an exemplary embodiment of the notifying section for notifying a result of evaluation by the evaluating section in the present invention.

Next, a third variation will be described below. The first scan planes SP1 may be defined without an ultrasonic scan performed by the first operator OP1. In this case, in place of an ultrasonic scan by the first operator OP1, for example, definition of the first scan planes SP1 may be performed at the ultrasonic diagnostic apparatus 1 or an apparatus such as a workstation separate from ultrasonic diagnostic apparatus 1.

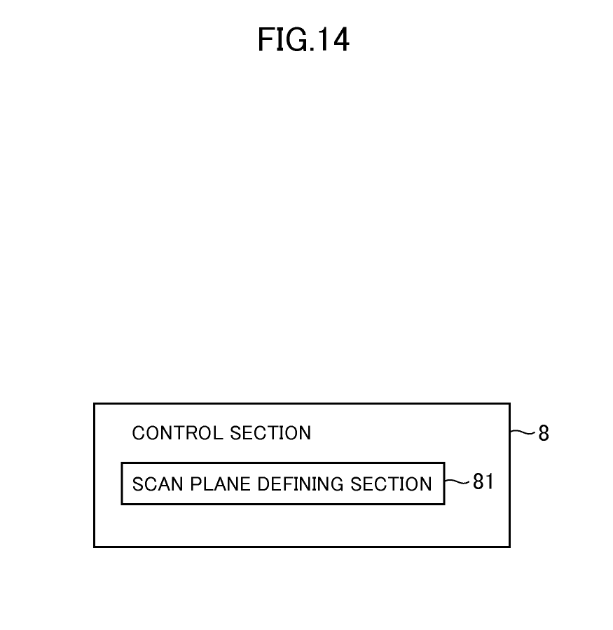

A case in which definition of the first scan plane SP1 is performed at the ultrasonic diagnostic apparatus 1 will be exemplarily described below. As shown in FIG. 14, the control section 8 executes a function of a scan plane defining section 81 by a program.

An operator makes an input at the operating section 7 for defining a 3D region for which an ultrasonic scan is to be performed. For example, an image representing an outline of the 3D region may be displayed in the display section 6, and the 3D region may be defined by the operator making an input for adjusting the image at the operating section 7. The 3D region is a region for which an imaginary first 3D ultrasonic scan is performed by a skilled operator. Once the imaginary 3D region has been thus defined, the scan plane defining section 81 defines first scan planes SP1 in the 3D region. For example, the first scan planes SP1 are defined at specified and equal intervals, as in the case in which the ultrasonic probe 2 is moved by a skilled operator.

The information on positions of the first scan planes SP1 defined by the scan plane defining section 81 is stored in the storage section 9. The information on positions of the first scan planes SP1 is information on a relative positional relationship among the first scan planes SP1.

When the first image G1 is displayed based on the information on positions thus stored in the storage section 9, the image display control section 53 displays the first scan plane SP1A and second scan plane SP2A at the same positions in the display section 6. The image display control section 53 also displays the other first scan plane SP1B, SP1C, SP1D based on the relative positional relationship among the first scan planes SP1A-SP1D identified from the information on positions stored in the storage section 9, and displays the other second scan planes SP2B, SP2C, SP2D based on the relative positional relationship among the second scan planes SP2A-SP2D identified from the information on positions obtained at the position identifying section 51.

### Second Embodiment

Next, a second embodiment will be described below. It should be noted that similar components to those in the first embodiment are designated by similar symbols and explanation thereof will be omitted.

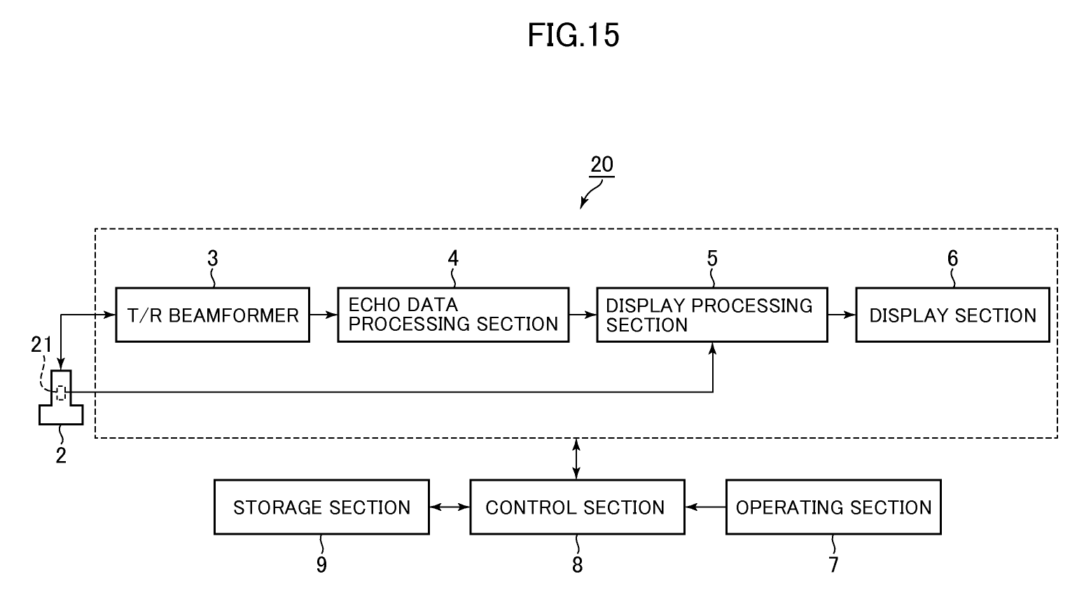

An ultrasonic diagnostic apparatus 20 in the present embodiment has the ultrasonic probe 2 provided with an acceleration sensor 21, as shown in FIG. 15. The acceleration sensor 21 may be provided in the inside of a housing, for example, constituting the ultrasonic probe 2. Detection signals from the acceleration sensor 21 are input to the display processing section 5. The acceleration sensor 21 represents an exemplary embodiment of the acceleration sensor in the present invention.

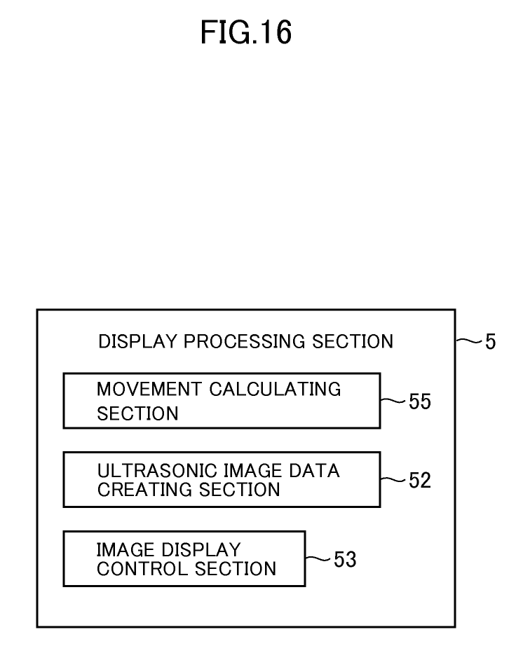

In the present embodiment, the display processing section 5 has a movement calculating section 55 in place of the position identifying section 51, as shown in FIG. 16. The movement calculating section 55 represents an exemplary embodiment of the movement calculating section in the present invention.

The movement calculating section 55 calculates movement of the ultrasonic probe 2 based on the detection signals from the acceleration sensor 21. In particular, the movement calculating section 55 calculates a movement distance and/or a movement direction of the ultrasonic probe 2. In case that a positional relationship between the acceleration sensor 21 and an ultrasonic scan plane is identified beforehand, calculation of movement of the ultrasonic probe 2 causes movement of the ultrasonic scan plane to be calculated. Thus, positions of the ultrasonic scan planes at certain intervals of time are identified.

In the present embodiment, the image display control section 53 displays the second image G2 based on the information on movement of the scan plane calculated by the movement calculating section 55. In the present embodiment, again, the position of the first scan plane SP1A, which is the scan-start plane among a plurality of the first scan planes SP1, and that of the second scan plane SP2A, which is the scan-start plane among a plurality of the second scan planes SP2, are assumed to be identical in the subject. The image display control section 53 displays the first scan plane SP1A and second scan plane SP2A at the same positions in the display section 6. The image display control section 53 also displays the other first scan planes SP1B, SP1C, SP1D based on a relative positional relationship among the first scan planes SP1A-SP1D identified from the information on positions stored in the storage section 9, and displays the other second scan planes SP2B, SP2C, SP2D based on a relative positional relationship among the second scan planes SP2A-SP2D identified from the information on positions obtained by the movement calculating section 51.

According to the present embodiment described above, similarly to the first embodiment, the first scan planes SP1 and second scan planes SP2 are displayed so that they have mutually corresponding positional relationships, and therefore, a similar effect to that in the first embodiment can be obtained.

It should be noted that the present embodiment may be practiced with several modifications as in the variations of the first embodiment.

### Third Embodiment

Next, a third embodiment will be described below. It should be noted that similar components to those in the first and second embodiments are designated by similar symbols and explanation thereof will be omitted.

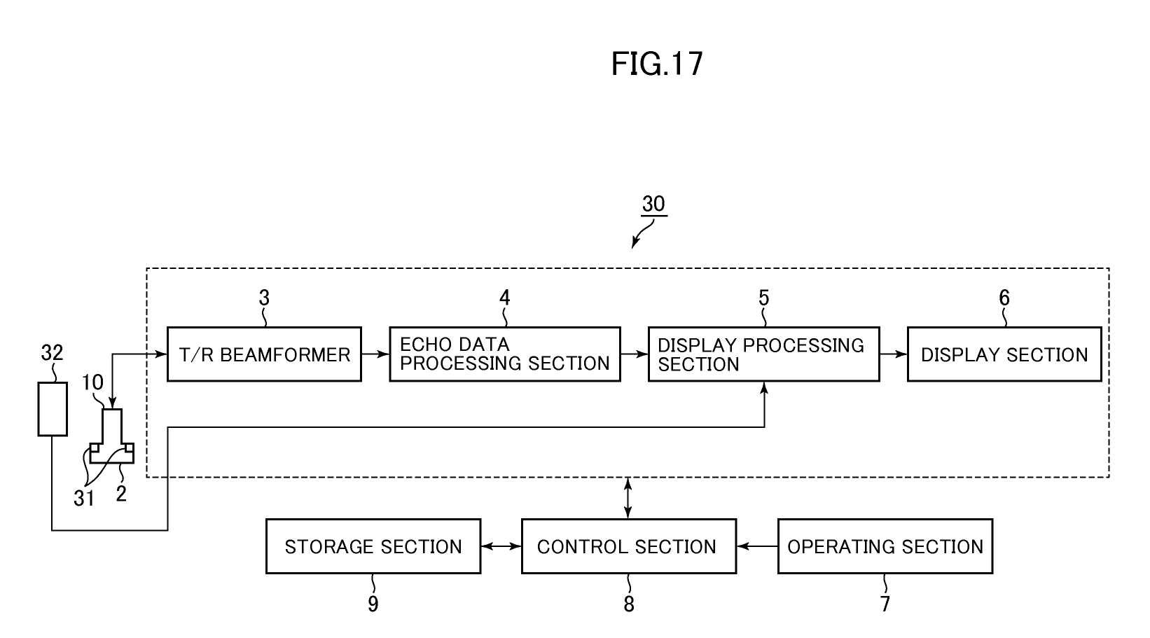

An ultrasonic diagnostic apparatus 30 in the present embodiment has the ultrasonic probe 2 provided with a light emitting section 31, as shown in FIG. 17. The light emitting section 31 emits light having a wavelength of visible light or of non-visible light such as infrared rays. The light emitting section 31 may be provided on a surface of a housing, for example, constituting the ultrasonic probe 2. The light emitting section 31 represents an exemplary embodiment of the light emitting section in the present invention.

Light from the light emitting section 31 is detected by a camera 32. Detection signals from the camera 32 are input to the display processing section 5. The camera 31 represents an exemplary embodiment of the light detecting section in the present invention.

In the present embodiment, again, the display processing section 5 has the movement calculating section 55. In the present embodiment, the movement calculating section 55 calculates a movement distance and/or a movement direction of the ultrasonic probe 2 based on the detection signals from the camera 32. In particular, the movement calculating section 55 calculates the movement distance and/or movement direction of the ultrasonic probe 2 according to movement of light emitted from the surface of the ultrasonic probe 2.

In the present embodiment, the first image G1 and second image G2 are displayed in a similar manner to that in the second embodiment. Therefore, a similar effect to that in the first and second embodiments can be obtained by the present embodiment.

It should be noted that the present embodiment may also be practiced with several modifications as in the variations of the first embodiment.

While the present invention has been described with reference to the embodiments, it will be easily recognized that the present invention may be practiced with several modifications without departing from the spirit and scope thereof. For example, when an ultrasonic scan plane by the second operator OP2 is not included in the first 3D region comprised of a plurality of the first scan planes SP1, the display image control section 53 may display a warning image comprised of characters to that effect in the display section 6. In this case, the display image control section 53 displays the warning image in the display section 6 based on information on positions in the storage section 9 and information on positions obtained by the position identifying section 51 or movement calculating section 55. However, this applies only to a case in which the position of the subject P is unchanged between a first 3D ultrasonic scan by the first operator OP1 and a second 3D ultrasonic scan by the second operator OP2, or a case in which the position of the first scan plane SP1A, which is the scan-start plane among a plurality of the first scan planes SP1, and that of the second scan plane SP2A, which is the scan-start plane among a plurality of the second scan planes SP2, are identical in the subject.

Moreover, the movement calculating section 55 may calculate a distance between different scan planes based on a correlation between ultrasonic images in different ultrasonic scan planes, instead of the calculation of movement of the ultrasonic probe 2 based on the detection signals from the acceleration sensor 21 or camera 32. Unlike the embodiments described above, this applies only to a case in which a 3D ultrasonic scan is performed while the ultrasonic probe 2 is translated.

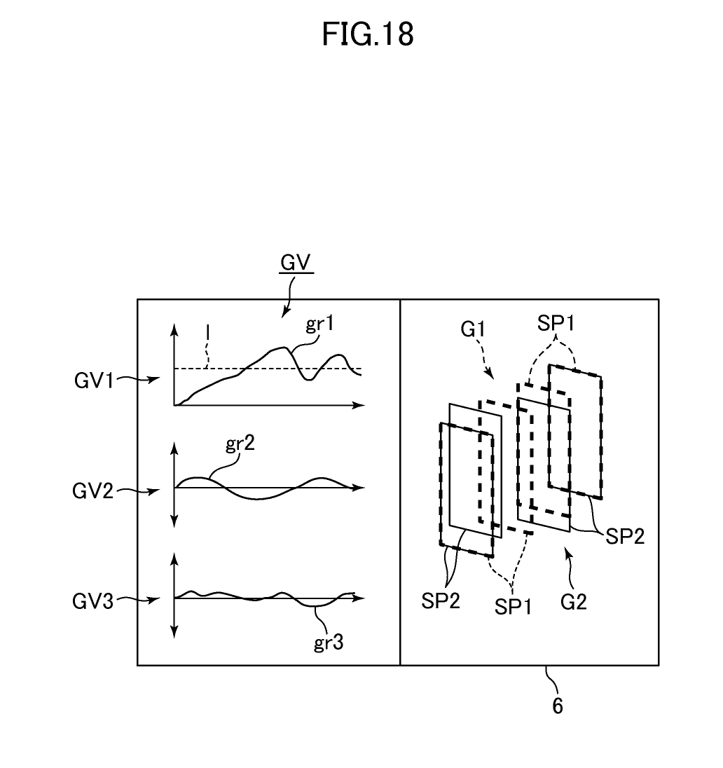

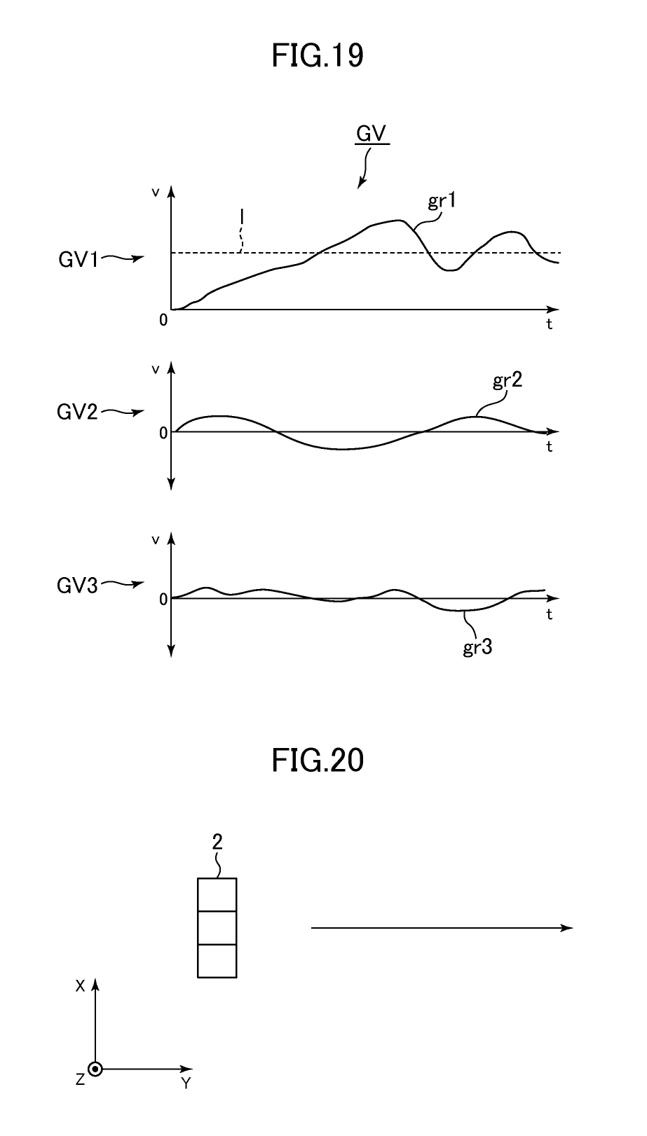

Furthermore, the image display control section 53 may display velocity variation images GV representing a temporal change of the velocity of movement of the ultrasonic probe 2 in the display section 6, as shown in FIG. 18. The velocity variation images GV are displayed along with the first image G1 and second image G2. The velocity variation images GV include a first velocity variation image GV1, a second velocity variation image GV2, and a third velocity variation image GV3. The first velocity variation image GV1, second velocity variation image GV2, and third velocity variation image GV3 each have a horizontal axis of time t and a vertical axis of velocity v, as shown in FIG. 19 in an enlarged view.

The velocity variation images GV represent a temporal change of the velocity of movement of the ultrasonic probe 2 when it moves in a Y-axis direction on a plane of abutment on the subject to form a plurality of scan planes in the Y-axis direction, as shown in FIG. 20. The movement of the ultrasonic probe 2 as shown in FIG. 20 gives a plurality of scan planes parallel to one another. Therefore, in the first image G1 and second image G2, each of a plurality of scan planes SP1 and each of a plurality of scan planes SP2 are displayed to be in parallel to each other.

The first velocity variation image GV1 shows a temporal change of the velocity of movement of the ultrasonic probe 2 in the Y-axis direction. The second velocity variation image GV2 shows a temporal change of the velocity of movement of the ultrasonic probe 2 in an X-axis direction. The third velocity variation image GV3 shows a temporal change of the velocity of movement of the ultrasonic probe 2 in a Z-axis direction.

The X-axis direction is an azimuthal direction of the ultrasonic probe 2. The Y-axis direction is an elevational direction of the ultrasonic probe 2. The Z-axis direction is a direction in which ultrasound is transmitted.

The first velocity variation image GV1 includes a first graph gr1. The second velocity variation image GV2 includes a second graph gr2. The third velocity variation image GV3 includes a third graph gr3. The first graph gr1, second graph gr2, and third graph gr3 represent a temporal change of the velocity of movement of the ultrasonic probe 2 when the second operator OP2 is moving the ultrasonic probe 2, for example.

The first velocity variation image GV1 also includes a straight line 1, represented by a dashed line, in a horizontal direction. The straight line 1 represents a temporal change of the velocity of ideal movement of the ultrasonic probe 2 by a skilled operator. In the second velocity variation image GV2, the horizontal axis representing a velocity of zero represents ideal movement of the ultrasonic probe 2 by the skilled operator without wobble of the ultrasonic probe 2 in the X-axis direction. In the third velocity variation image GV3, the horizontal axis representing a velocity of zero represents ideal movement of the ultrasonic probe 2 by the skilled operator without movement of the ultrasonic probe 2 in the Z-axis direction.

However, no straight line 1 may be displayed. Moreover, no first images G1 may be displayed.

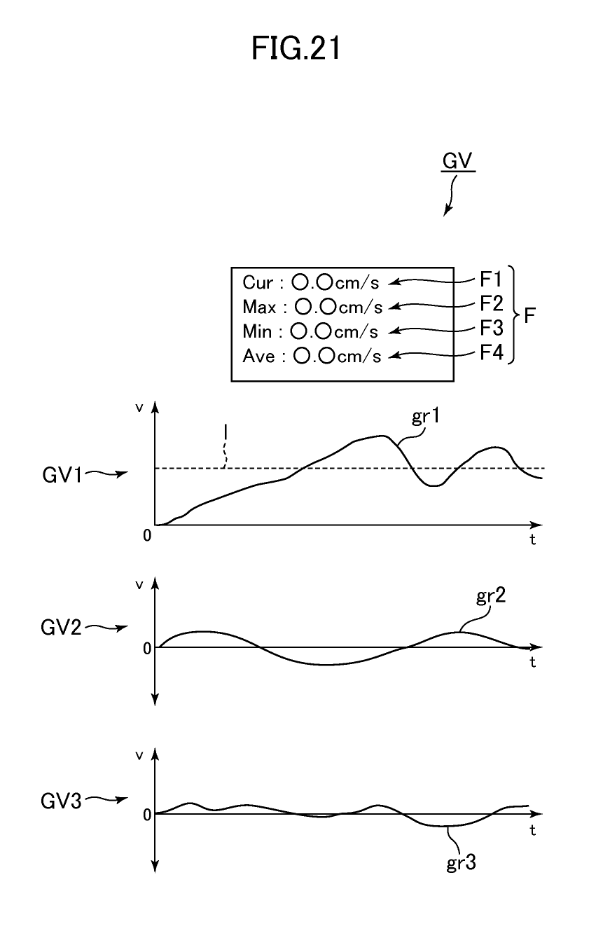

The velocity variation images GV may include numeric values indicating the velocity of movement of the ultrasonic probe 2. For example, the velocity variation images GV shown in FIG. 21 include numeric values F indicating the velocity of the ultrasonic probe 2 in the Y-axis direction. The numeric values F are a numeric value F1 (in a field of “Cur” in FIG. 21) indicating a current velocity of the ultrasonic probe 2, a numeric value F2 (in a field of “Max” in FIG. 21) indicating a maximum velocity of the ultrasonic probe 2 from the start of the movement of the ultrasonic probe 2 to the present, a numeric value F3 (in a field of “Min” in FIG. 21) indicating a minimum velocity of the ultrasonic probe 2 from the start of the movement of the ultrasonic probe 2 to the present, and a numeric value F4 (in a field of “Ave” in FIG. 21) indicating an average velocity of the ultrasonic probe 2 from the start of the movement of the ultrasonic probe 2 to the present. The velocities of the ultrasonic probe 2 in the X- and Z-axis directions may be displayed in numeric values.

By thus displaying the first velocity variation image GV1, second velocity variation image GV2, and third velocity variation image GV3, an inexperienced operator can move the ultrasonic probe 2 like a skilled operator by moving the ultrasonic probe 2 so that the first graph gr1 aligns with the straight line 1, the second graph gr2 aligns with the horizontal axis, and the third graph gr3 aligns with the horizontal axis.

### DESCRIPTION OF REFERENCE SYMBOLS

1, 20, 30 Ultrasonic diagnostic apparatus
2 Ultrasonic probe
6 Display section
9 Storage section
10 Magnetic sensor
11 Magnetism generating section
21 Acceleration sensor
31 Light emitting section
32 Camera
51 Position identifying section
53 Image display control section
54 Evaluating section
55 Movement calculating section

What is claimed is:

1. An ultrasonic diagnostic apparatus, comprising:
an ultrasonic probe for performing transmission/reception of ultrasound on a subject;
a movement detecting section for detecting movement of an ultrasonic scan plane by said ultrasonic probe;
a storage section for storing therein information on positions of a plurality of first scan planes in a first three-dimensional (3D) ultrasonic scan on said subject; and
an image display control section for displaying in a display section a first image representing positions of said first scan planes based on said information on positions stored in said storage section, and displaying in said display section, based on detected information from said movement detecting section, a second image representing positions of a plurality of second scan planes formed by performing a second 3D ultrasonic scan on said subject by an operator moving said ultrasonic probe, said image display control section displaying said first and second images so that said first scan planes and second scan planes have mutually corresponding positional relationships.

2. The ultrasonic diagnostic apparatus as recited in claim 1, wherein said first 3D ultrasonic scan is a scan performed on said subject by moving said ultrasonic probe separately from said second 3D ultrasonic scan, and said information on positions of said first scan planes is identified based on detection information from said movement detecting section when said first 3D ultrasonic scan is performed by said ultrasonic probe.

3. The ultrasonic diagnostic apparatus as recited in claim 1, wherein said first 3D ultrasonic scan is an imaginary ultrasonic scan, and said information on positions of said first scan planes is information defined and stored in said storage section beforehand.

4. The ultrasonic diagnostic apparatus as recited in claim 1, wherein:
said movement detecting section is configured to detect positions of said second scan planes in a coordinate system whose origin lies at a specified point,
said information on positions of said first scan planes is information on positions in said coordinate system whose origin lies at a specified point, and
said image display control section displays said first and second images based on positions of said first scan planes and second scan planes identified in said coordinate system whose origin lies at a specified point.

5. The ultrasonic diagnostic apparatus as recited in claim 1, wherein said image display control section is configured to:
display at the same position in said display section a first scan-start plane, which is a scan-start plane among a plurality of said first scan planes, and a second scan-start plane, which is a scan-start plane among a plurality of said second scan planes, lying at an identical position in said subject to that of said first scan-start plane,
display said first scan planes other than said first scan-start plane based on a relative positional relationship among a plurality of said first scan planes identified from said information on positions in said storage section, and
display said second scan planes other than said second scan-start plane based on a relative positional relationship among a plurality of said second scan planes identified from said detection information from said movement detecting section.

6. The ultrasonic diagnostic apparatus as recited in claim 1, further comprising:
an evaluating section for evaluating movement of said ultrasonic probe based on a positional relationship between said first and second scan planes corresponding in time to each other; and
a notifying section for notifying a result of evaluation by said evaluating section.

7. The ultrasonic diagnostic apparatus as recited in claim 4, further comprising:
an evaluating section for evaluating movement of said ultrasonic probe based on a positional relationship between said first and second scan planes corresponding in time to each other; and
a notifying section for notifying a result of evaluation by said evaluating section.

8. The ultrasonic diagnostic apparatus as recited in claim 5, further comprising:
an evaluating section for evaluating movement of said ultrasonic probe based on a positional relationship between said first and second scan planes corresponding in time to each other; and
a notifying section for notifying a result of evaluation by said evaluating section.

9. The ultrasonic diagnostic apparatus as recited in claim 1, wherein said movement detecting section comprises a magnetism generating section, and a magnetism detecting section is provided in said ultrasonic probe for detecting magnetism from said magnetism generating section, the ultrasonic diagnostic apparatus further comprising:
a position identifying section for identifying a position of said scan plane in a coordinate system whose origin lies at said magnetism generating section based on a magnetism detection signal from said magnetism detecting section.

10. The ultrasonic diagnostic apparatus as recited in claim 1, wherein said movement detecting section comprises an acceleration sensor provided in said ultrasonic probe, the ultrasonic diagnostic apparatus further comprising:
a movement calculating section for calculating movement of said scan plane based on detection signal from said acceleration sensor.

11. The ultrasonic diagnostic apparatus as recited in claim 1, wherein said movement detecting section comprises a light emitting section provided in said ultrasonic probe for emitting light having a wavelength of visible or non-visible light, the ultrasonic diagnostic apparatus further comprising:
a movement calculating section for calculating movement of said scan plane based on a detection signal from a light detecting section for detecting light from said light emitting section.

12. The ultrasonic diagnostic apparatus as recited in claim 1, wherein said movement detecting section detects a distance between different ultrasonic scan planes based on a correlation between ultrasonic images in said different ultrasonic scan planes.

13. The ultrasonic diagnostic apparatus as recited in claim 1, wherein:
said first image comprises a first 3D region image indicating a 3D region for said first 3D ultrasonic scan, and
said second image comprises a second 3D region image indicating a 3D region on which said second 3D ultrasonic scan is performed.

14. The ultrasonic diagnostic apparatus as recited in claim 4, wherein:
said first image comprises a first 3D region image indicating a 3D region for said first 3D ultrasonic scan, and
said second image comprises a second 3D region image indicating a 3D region on which said second 3D ultrasonic scan is performed.

15. The ultrasonic diagnostic apparatus as recited in claim 5, wherein:
said first image comprises a first 3D region image indicating a 3D region for said first 3D ultrasonic scan, and
said second image comprises a second 3D region image indicating a 3D region on which said second 3D ultrasonic scan is performed.

16. The ultrasonic diagnostic apparatus as recited in claim 13, further comprising:
a warning notifying section for notifying a warning when a second 3D region indicated by said second 3D region image is smaller than a first 3D region indicated by said first 3D region image.

17. The ultrasonic diagnostic apparatus as recited in claim 14, further comprising:
a warning notifying section for notifying a warning when a second 3D region indicated by said second 3D region image is smaller than a first 3D region indicated by said first 3D region image.

18. The ultrasonic diagnostic apparatus as recited in claim 13, further comprising:
a guide information notifying section for notifying, when said second 3D region is smaller than said first 3D region and an unscanned region for which said second 3D ultrasonic scan is not performed exists in said first 3D region, a guide information for guiding an ultrasonic scan by said ultrasonic probe to said unscanned region.

19. The ultrasonic diagnostic apparatus as recited in claim 13, further comprising:
a warning notifying section for notifying a warning when said second scan plane is not included in said first 3D region.

20. An ultrasonic diagnostic apparatus, comprising:
an ultrasonic probe for performing transmission/reception of ultrasound on a subject;
a movement detecting section for detecting movement of an ultrasonic scan plane by said ultrasonic probe;
a storage section for storing therein information on positions of a plurality of first scan planes in a first three-dimensional (3D) ultrasonic scan on said subject; and
a processor comprising an image display control function of displaying in a display section a first image representing positions of said first scan planes based on said information on positions stored in said storage section, and displaying in said display section, based on detected information from said movement detecting section, a second image representing positions of a plurality of second scan planes formed by performing a second 3D ultrasonic scan on said subject by an operator moving said ultrasonic probe, said image display control function displaying said first and second images so that said first scan planes and second scan planes have mutually corresponding positional relationships.
